(19) United States
(12) Patent Application Publication
(10) Pub. No.: US 2015/0177208 A1
(43) Pub. Date: Jun. 25, 2015

(54) GAS SENSOR WITH INTERFACE TO HAND-HELD INSTRUMENT

(71) Applicant: Jonathan David Murphy, Friday Harbor, WA (US)

(72) Inventor: Jonathan David Murphy, Friday Harbor, WA (US)

(21) Appl. No.: 14/525,449

(22) Filed: Oct. 28, 2014

Related U.S. Application Data
(63) Continuation-in-part of application No. 14/136,643, filed on Dec. 20, 2013.
Application No. 14/136,643 is a continuation-in-part of application No. 14/502,142, filed on Sep. 30, 2014.

(51) Int. Cl. G01N 33/00

(52) U.S. Cl. CPC G01N 33/0063

(57) ABSTRACT

A sensory system, apparatus, and method for gas concentration measurement. The sensory apparatus includes a gas sensor, and an identification reader configured to acquire data representing an identifier from an identification tag. The apparatus also includes a controller coupled with the gas sensor and the identification reader. In response to the identification reader acquiring the data representing the identifier, the controller causes the gas sensor to measure a concentration of a gas in a test area.

### CROSS-REFERENCE TO RELATED APPLICATIONS

This application is a continuation-in-part of U.S. patent application Ser. No. 14/136,643, which was filed on Dec. 20, 2013, and a continuation-in-part of U.S. patent application Ser. No. 14/502,142, filed on Sep. 30, 2014. The entirety of these priority applications is incorporated herein by reference.

### BACKGROUND

Gas detectors or “sniffers” measure and indicate the presence and/or concentration of certain gases in an air. Such sniffers are employed to prevent toxic exposure and/or fire, and may be required to ensure regulatory compliance. Gas sniffers may be configured to detect one type of gas (e.g., carbon monoxide, chlorine gas, nitrogen oxides, etc.) or may be capable of detecting several gases. A variety of gas-sensing technologies are available, including electrochemical sensors, infrared sensors, metal oxide semiconductors, catalytic sensors, and infrared sensors.

Some gas sensors are stationary, e.g., mounted to a floor or wall, while others may be provided in compact, portable packages, which may facilitate mobile use. However, the advantages to such portability of these sensors are often limited by computing power, battery life, and display size. One way to address these challenges is to provide more powerful and efficient processors, higher-capacity batteries, and higher-resolution displays. However, a conservation of these resources may be beneficial, for example, from a cost standpoint. Moreover, different types of sensors may be called for in taking different types of measurements (i.e., a thickness measurement may call for an ultrasonic thickness gauge, rather than a gas sensor). Thus, users are often forced to carry several different sensors when, for example, conducting a walk-around inspection of an industrial facility.

In addition, certain locations may be susceptible to exposure to different gases, e.g., according to the operations being conducted and/or materials being handled. Further, different areas may be more or less sensitive to the concentration of certain gases, and thus a static alarm or “threshold” level of gas concentration may not be uniformly suitable. Moreover, a recorded history, e.g., for proving regulatory compliance, may be needed, and may be tied to particular locations or rooms within a facility. Accurately recording and tracking such information, while conducting potentially several other measurements may thus become cumbersome.

### SUMMARY

Embodiments of the disclosure may provide a sensory apparatus for gas concentration measurement. The sensory apparatus includes a gas sensor, and an identification reader configured to acquire data representing an identifier from an identification tag. The apparatus also includes a controller coupled with the gas sensor and the identification reader. In response to the identification reader acquiring the data representing the identifier, the controller causes the gas sensor to measure a concentration of a gas in a test area.

Embodiments of the disclosure may also provide a sensor system. The sensor system includes a mobile unit having a power source, and a sensory head releasably coupled with the mobile unit, such that the sensory head is in electrical communication with the mobile unit so as to receive power from the power source and to provide a communication signal to the mobile unit. The sensory head includes a gas sensor, and an identification reader configured to acquire data representing an identifier from an identification tag. The sensory head also includes a controller coupled with the gas sensor and the identification reader. In response to the identification reader acquiring the data representing the identifier, the controller causes the gas sensor to measure a concentration of a gas in a test area.

Embodiments of the disclosure further include a method for measuring a gas concentration. The method includes coupling a sensory head to a mobile unit such that the sensory head receives at least power from the mobile unit and supplies a communication signal to the mobile unit. The sensory head includes a controller, an identification reader coupled with the controller, and a gas sensor coupled with the controller. The method also includes obtaining data representing an identifier from an identification tag positioned in, on, or proximal to a test area, using the identification reader, and in response to obtaining the data representing the identifier, taking one or more gas concentration measurements using the gas sensor.

### BRIEF DESCRIPTION OF THE DRAWINGS

The accompanying drawings, which are incorporated in and constitutes a part of this specification, illustrates an embodiment of the present teachings and together with the description, serves to explain the principles of the present teachings. In the figures:

FIG. 1 illustrates a schematic view of a sensor system, according to an embodiment.

FIG. 2 illustrates an exploded, perspective view of a sensory head, according to an embodiment.

FIG. 3 illustrates an exploded perspective view of contacts and an isolator ring of the sensory head, according to an embodiment.

FIG. 4 illustrates a perspective view of a shell body of the sensory head, according to an embodiment.

FIG. 5 illustrates a perspective view of a controller of the sensory head, according to an embodiment.

FIG. 6 illustrates an exploded, perspective view of a gas sensor and a hood of the sensory head, according to an embodiment.

FIG. 7 illustrates a flowchart of a method for sensing a gas concentration in an area, according to an embodiment.

It should be noted that some details of the figures have been simplified and are drawn to facilitate understanding of the embodiments rather than to maintain strict structural accuracy, detail, and scale.

### DETAILED DESCRIPTION

Reference will now be made in detail to embodiments of the present teachings, examples of which are illustrated in the accompanying drawing. In the drawings, like reference numerals have been used throughout to designate identical elements, where convenient. In the following description, reference is made to the accompanying drawing that forms a part thereof, and in which is shown by way of illustration a specific exemplary embodiment in which the present teachings may be practiced. The following description is, therefore, merely exemplary.

In general, embodiments of the present disclosure provide a mobile unit that is electrically and mechanically coupled with a sensory head. The sensory head provides a gas sensor, such as a three-wire electrochemical cell, as well as an identification reader, such as a radio-frequency identification (RFID) tag reader, to name one example. The reader may be configured to acquire an identifier (e.g., tag number) from an identification tag (e.g., RFID tag) positioned at a predetermined location in, on, or near to a test area. The identifier may be linked in a database to acceptable gas levels, gases to test for, historical gas tests, etc., which may be related to the predetermined location of the identification tag. The mobile unit, or a remote computing device (e.g., a ruggedized, hand-held computing device) in communication with the mobile unit, may access the location-specific information, which it may use to calibrate or otherwise analyze measurements taken by the sensory head.

Furthermore, the sensory head may be configured to automatically take gas concentration measurements in response to reading an identifier from the identification tag. The data collected by the gas sensor may be transmitted to the mobile unit and/or the remote device for displaying, processing, storage, etc., e.g., in association with the identifier.

As noted above, the mobile unit may be provided with an electro-mechanical interface with the sensory head. The interface may be configured to support a connection with one or more other, different types of sensors, thereby, for example, providing a modular extensibility for the mobile unit. As such, the interface may allow a user to invest in one portable instrument with multiple sensor heads, which may save money and may facilitate walk-around data collection. Furthermore, the mobile unit may interface with software applications that allow for a consistent user interaction and data sharing across the organizations, while receiving and analyzing input from different types of sensory heads.

Moreover, one advantage of having the sensor and identification reader close together (e.g., co-located in the sensory head) is that the identification tag may be mounted at the measurement location, and the identification tag may be scanned to start the measurement. The sensory head may thus be able to be fed data representing the general physical location of the accessory sensor head and the logical measurement settings and alarm criteria associated with the measurement location. It will be appreciated that these advantages and/or others may be provided in various embodiments of the present disclosure; however, these advantages should not be considered limiting or in any way otherwise required.

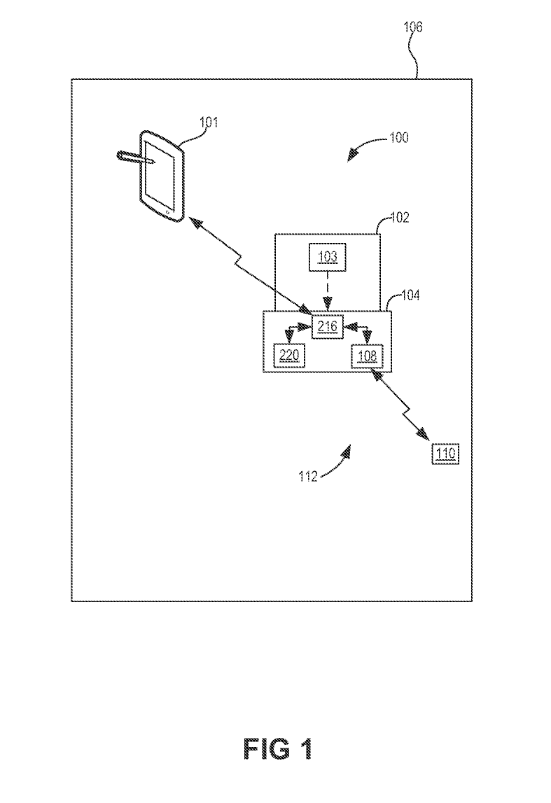

Turning now to the Figures, FIG. 1 illustrates a schematic, perspective view of a sensor system 100, according to an embodiment. The sensor system 100 may include a mobile unit 102 that is in communication with a remote device 101. The mobile unit 102 may include a power source 103, which may be a battery or a wired connection to an external power source, and may also include various other electrical and/or mechanical components.

The remote device 101 may be a portable electronic device, such as a smartphone, tablet, or laptop computer, or may be another type of specific or general-purpose computing device that is supplied with appropriate software. The mobile unit 102 may communicate with the remote device 101, and, in some embodiments, vice versa, via any suitable communications link, such as a wireless link (e.g., BLUETOOTH®, WiFi, WIMAX®, GSM, CDMA, LTE, etc.).

The sensor system 100 may also include a sensory apparatus or “head” 104 that may be releasably coupled, e.g., mechanically and electrically, with the mobile unit 102 so as to be positionally fixed thereto, receive at least power therefrom, and provide one or more signals thereto. The sensory head 104 may be a modular unit, which may be physically coupled with and easily removable from the mobile unit 102, such that the sensory head 104 and mobile unit 102 may be hand-held. When a different sensor is needed, the sensory head 104 may be removed from the mobile unit 102, and another sensory head may be attached. This may facilitate switching between different types of sensors, e.g., while testing areas of and/or various equipment in an industrial facility.

Further, in at least one embodiment, the mobile unit 102 may include a display screen, which may be configured to display data based on the gas concentration measurements taken using the sensory head 104. In other embodiments, the display screen may be omitted from the mobile unit 102, or may otherwise not display such data.

In the illustrated embodiment, the mobile unit 102 is depicted as being located, at least temporarily, in a test area 106. For example, the test area 106 may be a facility, such as an industrial facility, or a portion (e.g., room) thereof. The remote device 101 may be located in or outside of the test area 106. The test area 106 may represent a volume of air for which the concentration of one or more gases may be measured using the mobile unit 102, e.g., with the sensory head 104 attached thereto. Accordingly, the sensor system 100, e.g. the sensory head 104 of the mobile unit 102, may be configured to measure a gas concentration in the test area 106, e.g., in response to a trigger, as will be described in greater detail below.

Thus, the sensory head 104 may include, for example, a gas sensor 220. The gas sensor 220 may be any type of gas sensor, whether configured to detect one gas or several different types of gas concentrations. In one specific embodiment, the gas sensor 220 may include a three-wire electrochemical gas sensor; however, this is merely one example among many contemplated.

Further, the sensory head 104 may include an identification reader 108. The identification reader 108 may be configured to capture data representing an identifier, such as a tag number, and/or any other information from an identification tag 110 mounted in, on, or near to, or otherwise disposed proximal to, a location 112 in the test area 106.

In some embodiments, the identification reader 108 may be a radio-frequency identification (RFID) tag reader, and the identification tag 110 may be an active or passive RFID tag. As the term is used herein, “tag” broadly refers to any structure positionable at a location in, on, or near to a test area, and should not be interpreted as requiring any particular size or shape, unless otherwise expressly stated herein. In some embodiments, the tag 110 may be a small, thin structure made of one or several layers of material, which may be adhered to the location 112. In other embodiments, the identification tag 110 may be larger, include other components (such as a processor, display screen, other input and/or output peripherals, etc.), and/or may be incorporated into a larger device. In some embodiments, the identification tag 110 may display a QR code, whether statically (e.g., printed) or dynamically (e.g., displayed on a display screen). In another embodiment, the identification tag 110 may display a bar code, or may provide an identifier to the identification reader 108 through any other medium, such as sound, light (e.g., infrared) pulses, etc. The identification reader 108 may be suitably configured to read the QR code, bar code, or any other identifier transmission medium selected.

The identifier read from the identification tag 110 may be associated with location-specific information, e.g., in a table or database. The database may be stored on the mobile unit 102, on the remote device 101, or on another device communicably coupled with the mobile unit 102 and/or the remote device 101. For example, the location-specific information may include one or more gas composition alarm thresholds. Thus, the location-specific information may, in an embodiment, provide two types of information to the mobile unit 102: the type of gas for which the concentration is to be measured (i.e., the “relevant” gas composition) and the threshold alarm rate for that (or those) gases, above which a hazard may be indicated, e.g., via an alarm. In an embodiment, the location-specific information may additionally or instead represent historical gas compositions at the location 112 and/or otherwise in the test area 106. Such historical measurements may facilitate the calculation of measured gas composition trends and/or may provide quick-access to logs that may evidence historical regulatory compliance, trends, and/or effects of corrective/mitigating actions that may have been taken in response to previously-measured gas compositions. It will be appreciated that, in this context, “historical” refers to anything that has occurred in the past, but should not be considered to require any certain length of time in the past.

The sensory head 104 may also include a controller 216. As will be described in greater detail below, the controller 216 may be configured to communicate with the remote device 101, e.g., via a data-transmission device provided by the mobile unit 102. The communication between the controller 216 and the remote device 101 may be one or two-way. For example, the controller 216 may be configured to query a database accessible to the remote device 101 by communication with the remote device 101, may be configured to write to the database 101 by communication with the remote device 101, and may be configured to receive location-specific information, e.g., in response to a query, from the database accessible to the remote device 101.

The controller 216 may also communicate with the identification reader 108, so as to receive a signal indicative of a tag identifier therefrom. Further, the controller 216 may communicate with the gas sensor 220, so as to initiate and/or terminate operation thereof, as well as receive signals therefrom, which may be representative of data measured by the sensor 220. In some embodiments, the controller 216 may interface with another controller, e.g., in the mobile unit 102, to perform one or more of the foregoing functions.

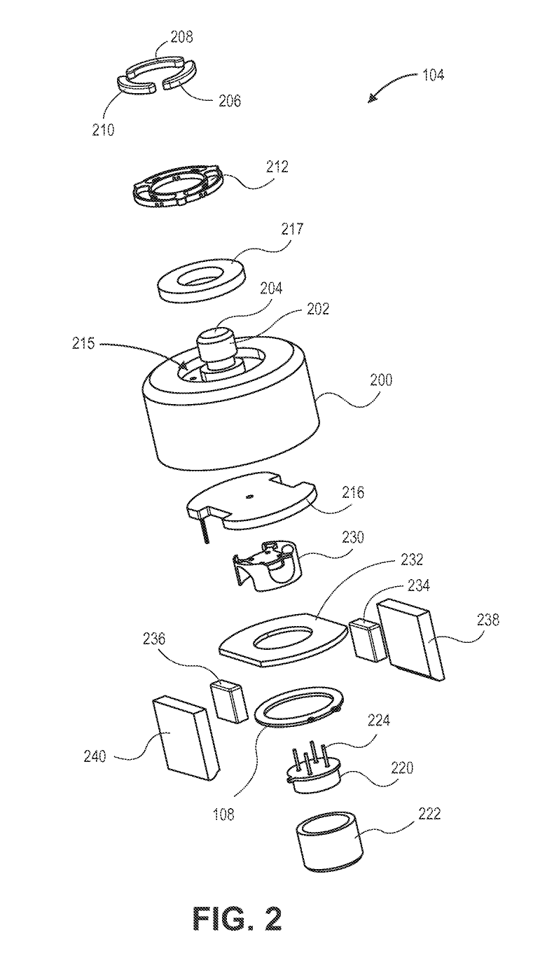

FIG. 2 illustrates an exploded view of the sensory head 104, according to an embodiment. It will be appreciated that the sensory head 104 displayed in FIG. 2 is but one specific example among many contemplated. As shown, the sensory head 104 includes a body shell 200, which may provide a rugged exterior for the sensory head 104. The sensory head 104 may also include a mounting device 202, such as a threaded stud, “quick-release” shaft, threaded opening, quick-release shaft collar, tabs, magnet, or any other device for mounting the sensory head 104 to the mobile unit 102. Accordingly, the mobile unit 102 (e.g., FIG. 1) may include a receptacle, such as a female threaded connection, quick connect shaft or collar, magnet, etc., for receiving and coupling with the mounting device 202.

The sensory head 104 may also include a ground connection 204, which may be integrated into the mounting device 202. The ground connection 204 may be electrically connected with the mobile unit 102. Accordingly, using the mounting device 202, the sensory head 104 may be placed into electro-mechanical communication with the mobile unit 102 and/or quickly removed from such communication therewith.

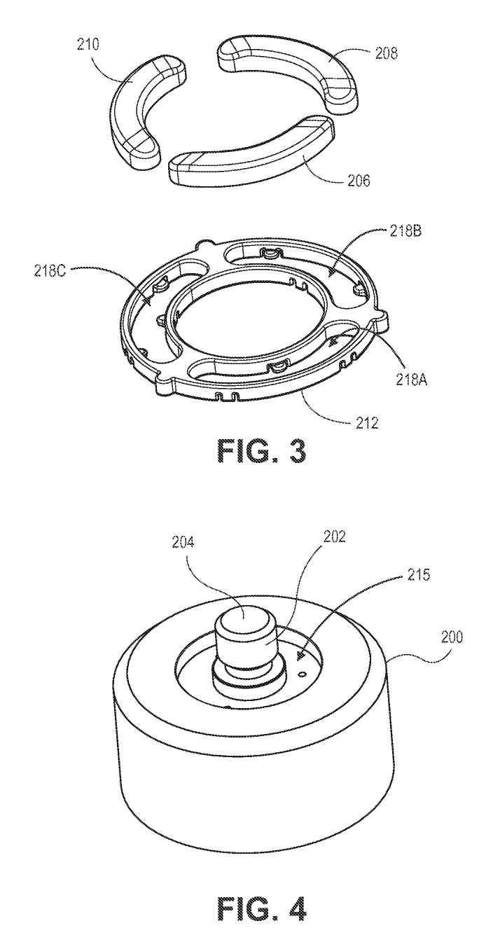

Several elements of the sensory head 104 embodiment depicted in FIG. 2 will now be described with reference to enlarged views thereof provided in FIGS. 3-6. Reference is thus made to each of FIGS. 3-6 individually, with continuing reference to the context provided by the illustrated embodiment of FIG. 2.

The sensory head 104 may include at least one contact (three are shown: 206, 208, 210) and an isolator ring 212. FIG. 3 illustrates an enlarged, perspective view of the contacts 206-210 and the isolator ring 212, according to an embodiment. As shown, the contacts 206-210 may be formed as arc-shaped members. The isolator ring 212 may include slots 218A, 218B, 218C, which may receive the contacts 206-210, respectively. The isolator ring 212 may thus serve to electrically isolate the contacts 206-210 from one another.

In an embodiment, the contact 206 may be configured to deliver power from the mobile unit 102 to the sensory head 104. The contact 208 may be configured to deliver a digital input signal from the mobile unit 102 to the sensory head 104. The contact 210 may be configured to deliver a digital output signal to the mobile unit 102, e.g., based on a signal from the sensory head 104. Although not depicted, in another embodiment, a fourth contact may be provided and seated within a slot of the isolator ring 212, for providing a communication signal from an identification reader (e.g., antenna) to the mobile unit 102, as will be described below.

FIG. 4 illustrates an enlarged, perspective view of the body shell 200, according to an embodiment. As mentioned above, the body shell 200 may include the mounting device 202 and the ground connection 204. The body shell 200 may also include a recess 215, which may be defined at least partially around the mounting device 202. The contacts 206-210 and the isolator ring 212 may seat in the recess 214; however, in other embodiments, the contacts 206-210 and body shell 200 may be coupled together, or otherwise positioned, in any other manner. Further, the contacts 206-210 and isolator ring 212 may be disposed in or “potted” in a resin 217, or another material, which may provide a stable and, for example, electrically-insulating mount for the contacts 206-210 and isolator ring 212 in the recess 215.

The sensory head 104 may further include a hood 222, which may cover at least a portion of the gas sensor 220, thereby shielding the gas sensor 220 from the surrounding environment. FIG. 6 illustrates an enlarged, exploded, perspective view of the sensor 220 and the hood 222, according to an embodiment. As shown, the sensor 220 may be physically coupled with at least one of a plurality of leads 224. For example, the sensor 220 may receive power and/or one or more digital communication signals via the leads 224 and provide a digital signal via another one of the leads 224. The leads 224 may, in turn, be electrically connected with the controller 216 and/or the contacts 206-210, and eventually with the mobile unit 102 (FIG. 1).

Referring now again specifically to FIG. 2, the sensory head 104 may also include the tag identification reader 108. The identification reader 108 may be configured to detect an identifier from the identification tag 110 (FIG. 1), as noted above. Accordingly, in an RFID embodiment, the identification reader 108 may be an RFID antenna (e.g., an active or passive RFID reader), and may include a tuned, wire loop, which may receive a modulated radio frequency signal from the RFID tag. The received radio frequency signal may provide data representing the identifier associated with the RFID tag. In active-reader embodiments, the identification reader 108 may also transmit a signal to the RFID tag, providing the energy for the signal carrying the identifier. In other embodiments, the identification reader 108 may be an optical sensor, which may read a QR code or a bar code provided by the identification tag 110, or may be any other type of sensor configured to receive information from the identification tag 110. In an embodiment, at least one of the contacts 206-210 may transmit the read tag information through a digital output signal, and out of the sensory head 104 into the mobile unit 102.

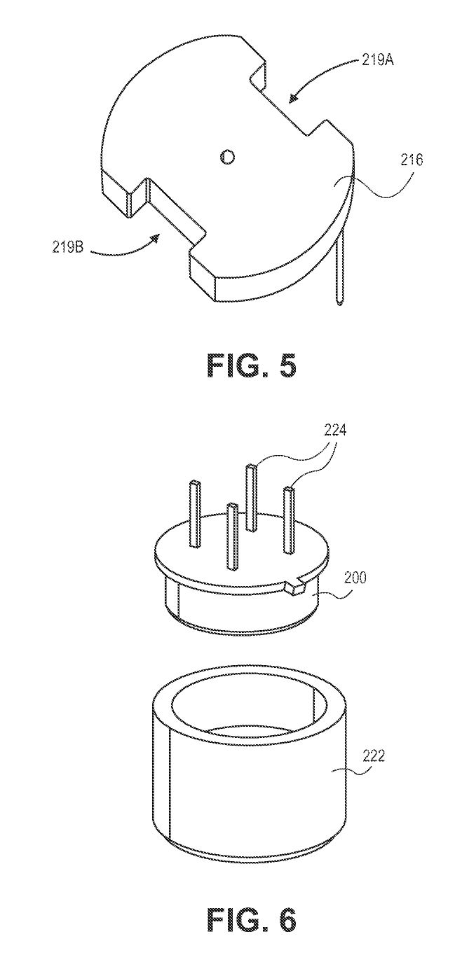

As mentioned above, the controller 216 may control operation of, including the power supply to, the sensory head 104, among other functions. FIG. 5 illustrates an enlarged view of the controller 216, according to an embodiment. The controller 216 may be or include one or more printed circuit boards (PCBs). In at least some embodiments, the controller 216 may include one or more microprocessors, electrical contacts/leads, electrical pathways, etc. The controller 216 may be in electrical communication with one or more of the contacts 206-210, as well as the ground connection 204.

Further, the controller 216 may define one or more magnet slots 219A, 219B, which may be formed as cut-outs extending inwards from the periphery of the controller 216. The controller 216 may be configured to selectively power the various elements of the sensory head 104, receive information therefrom, transmit information to the mobile unit 102, and/or otherwise control a functioning of the sensory head 104.

The sensory head 104 may also include a support assembly for the identification reader 108. For example, the support assembly may include a reader spacer 230 around which the identification reader 108 (e.g., a loop-shaped antenna) may be received, and a reader plate 232, which may couple with the identification reader 108 and the controller 216, so as to position the identification reader 108 with respect thereto. In some embodiments, however, the identification reader 108 may be or include an RFID spiral antenna or a wound inductor antenna, which may be coupled with the reader plate 232.

The sensory head 104 may also include one or more mounting feet (two are shown: 238, 240). The mounting feet 238, 240 may be fabricated at least partially of a highly-permeable material, which may transmit the magnetic flux generated by the magnets 234, 236. Further, the mounting feet 238, 240 may, in at least some embodiments, extend beyond the hood 222 so as to physically contact a test piece (e.g., a machine in the test area 106 which may be the subject of other measurements, such as thickness, temperature, vibration, etc. measurements, or any other structure). In other embodiments, the mounting feet 238, 240 may not extend past the hood 222 and may, instead, be housed therein, transmitting the magnetic flux therethrough. Moreover, it will be appreciated that the magnets 234, 236 and/or mounting feet 238, 240 may be provided in any suitable shape and configuration, with the illustrated embodiment being merely one among many contemplated. Furthermore, in some embodiments, the magnets 234, 236 may be configured to bear directly on a test piece (or another structure), with the mounting feet 238, 240 being omitted. In still other embodiments, the magnets 234, 236 and the mounting feet 238, 240 may be unnecessary and omitted.

Referring again to FIGS. 1 and 2, in an example of operation, the mobile unit 102 may be brought into proximity of the identification tag 110 (e.g., RFID tag). The identification reader 108 may read an identifier from the identification tag 110. For example, the identification reader 108 may poll for identification tags to read, e.g., by sending a signal constantly or intermittently. A preliminary trigger may also be provided to initiate such polling, such as a signal from the remote device 101, the controller 216 detecting the magnets 234, 236 engaging a test piece, or another trigger. In other embodiments, the identification reader 108 may be energized manually via user input to seek an identification tag 110 to read (e.g., to begin transmitting an energizing radio-frequency signal). In still other embodiments, the identification reader 108 may automatically detect proximity to an identification tag 110 in any other way.

The controller 216 may detect when the identification reader 108 reads a signal from the identification tag 110. In response, the controller 216 may cause the sensor 220 to begin the gas concentration measurement process. In a specific embodiment, the controller 216 may provide power to the sensor 220 and receive data therefrom, e.g., via the leads 224. In some embodiments, the controller 216 may be operable independently to cause the sensor 220 to commence measuring. In other embodiments, the controller 216 may interface with a separate controller housed in the mobile unit 102, the remote device 101, or elsewhere, for initiation of the gas concentration measuring.

In some embodiments, in addition to or instead of initiating measurement when the identification reader 108 reads an identifier, the controller 216 may be configured to detect when the magnets 234, 236 magnetically engage a test piece in the test area 106, e.g., via the mounting feet 238, 240. In some embodiments, the controller 216 (or another part of the mobile unit 102 and/or remote device 101) may be responsive to a user input, e.g., a user pressing a button on the mobile unit 102 and/or remote device 101, and may initiate gas concentration measurements in response to such manual input.

When the identification reader 108 obtains an identifier from the identification tag 110, the sensory head 104 (e.g., the controller 216) may provide a signal to the mobile unit 102 that includes a digital representation of the identifier. The mobile unit 102 may, in some embodiments, relay the identifier to the remote device 101. In some embodiments, the remote device 101 and/or the mobile unit 102 may query a database, using the obtained identifier, so as to determine the location-specific information associated with the test location 112 of the test area 106. The mobile unit 102 and/or the remote device 101 may then use this information to assist in calibration of sensor data, and/or analysis thereof.

For example, based at least partially on such location-specific information, the controller 216 may be configured to determine one or more gas-concentration thresholds. In an embodiment, the location-specific information may directly include such alarm thresholds. In another embodiment, the location-specific information may include a representation of a relative sensitivity level to one or more gases, and the controller 216 may apply one or more rules to determine the one or more alarm concentrations, based on the sensitivity levels. Further, the location-specific information may prescribe one or more specific gases that are relevant to the location 112, and thus the controller 216 may be configured to adjust the sensor 220 to measure for such gases, and/or may ignore portions of the signal from the gas sensor 220 related to concentration measurements for other types of gases.

Furthermore, the recorded measurement(s) from the sensor 220 may be stored in association with the identifier. For example, the controller 216 may be operable to write new location-specific information to the database (e.g., via the remote device 101 and/or the mobile unit 102). The identifier, which may be linked to the test area 106 and/or the specific location 112, may thus provide an index to a history of measurements, which may be updated each time one or more gas concentrations are measured for the test area 106 in response to a particular identifier being read.

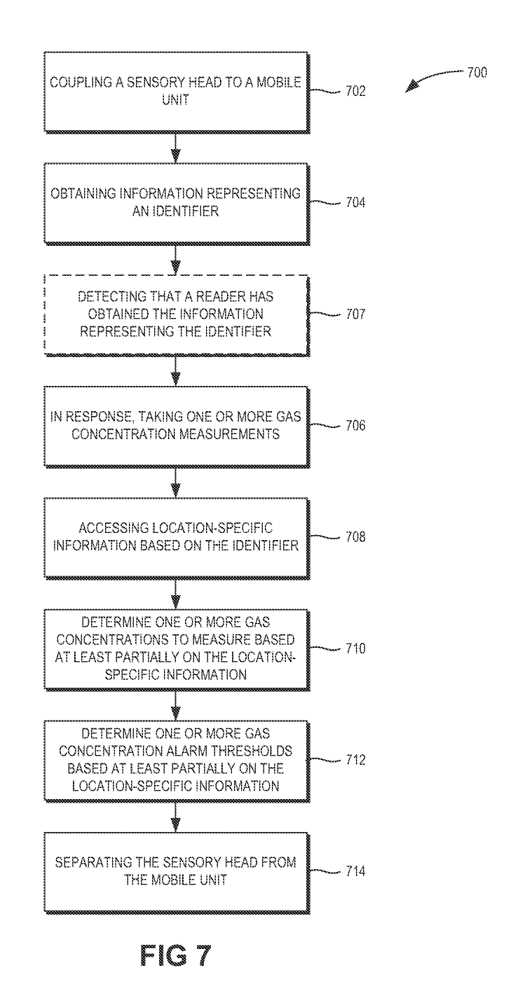

FIG. 7 illustrates a flowchart of a method 700 for measuring a gas concentration in a test area, according to an embodiment. The method 700 may proceed by operation of one or more embodiments of the sensor system 100 described above; however, at least some embodiments of the method 700 are not limited to any particular structure.

The method 700 may include coupling a sensory head to a mobile unit, as at 702. In such coupling, the sensory head may receive power and/or an input signal from the mobile unit. Further, the sensory head may supply a communication signal to the mobile unit. In some embodiments, the sensory head may include a controller, an identification reader coupled with the controller, and a gas sensor coupled with the controller.

The method 700 may also include obtaining data or information representing an identifier from an identification tag positioned on or proximal to a test location of a test piece, using the identification reader, as at 704. In one specific example, the identification tag may be an RFID tag, active or passive, which may be readable and/or writable by interface with the identification reader, which may be an RFID tag reader, whether active or passive. In other embodiments, other types of identification readers and tags may be employed.

The method 700 may further include, in response to obtaining the data representing the identifier, taking one or more gas concentration measurements using the gas sensor, as at 706. In an example, the controller may first detect when the identification reader has obtained the data representing the identifier, as at 707. The controller may then respond by activating (e.g., energizing) the gas sensor, so as to begin the gas concentration measurement. The controller may then, in at least some embodiments, receive a signal from the gas sensor, with the signal representing a gas concentration of one or more gasses in the test area. The controller may then relay the signal, or an interpretation thereof, to the mobile unit for further processing, display, and/or transmission to a remote device in communication (e.g., wirelessly) with the mobile unit.

In an embodiment, the method 700 may include accessing location-specific information related to the test location of the test piece based on the identifier, as at 708. For example, the location-specific information may be stored, in association with one or more identifiers, in a database that may be stored on the mobile unit or the remote device. The location-specific information may include, for example, an indication of one or more gases that the gas sensor should test for, a sensitivity of the test area associated with the identifier, a history of gas concentration measurements, and/or the like.

The method 700 may further include determining one or more gas concentrations (e.g., CO, CO2, NOX, etc.) for which concentration measurements may be taken in the test area, based on the location-specific information, as at 710. In other words, the location-specific information may provide data that may be interpreted as specifying a certain one or more types of gases to test for. In addition or instead, the method 700 may include determining one or more gas concentration or “alarm” thresholds, based at least in part on the location-specific information, as at 712. In an embodiment, the gas sensor may be caused to measure the concentration of the type of gas determined at 710 and/or may compare the gas concentration measurement with the gas concentration alarm threshold. The method 700 may thus also include determining that a hazard exists, e.g., when it is determined that the gas concentration exceeds the gas concentration threshold. In response, the method 700 may include indicating such a hazard, e.g., using an audible and/or visual, etc. alarm.

The method 700 may also include, in an embodiment, separating the sensory head from the mobile unit, as at 714. For example, the connection between the mobile unit and the sensory head may be releasable without damaging either. For example, multiple sensory heads, e.g., with multiple different types and/or sizes of sensors may be provided as modular units.

While the present teachings have been illustrated with respect to one or more implementations, alterations and/or modifications may be made to the illustrated examples without departing from the spirit and scope of the appended claims. In addition, while a particular feature of the present teachings may have been disclosed with respect to only one of several implementations, such feature may be combined with one or more other features of the other implementations as may be desired and advantageous for any given or particular function. Furthermore, to the extent that the terms “including,” “includes,” “having,” “has,” “with,” or variants thereof are used in either the detailed description and the claims, such terms are intended to be inclusive in a manner similar to the term “comprising.” Further, in the discussion and claims herein, the term “about” indicates that the value listed may be somewhat altered, as long as the alteration does not result in nonconformance of the process or structure to the illustrated embodiment. Finally, “exemplary” indicates the description is used as an example, rather than implying that it is an ideal.

Other embodiments of the present teachings will be apparent to those skilled in the art from consideration of the specification and practice of the present teachings disclosed herein. It is intended that the specification and examples be considered as exemplary only, with a true scope and spirit of the present teachings being indicated by the following claims.

What is claimed is:

1. A sensory apparatus for gas concentration measurement, comprising:
a gas sensor;
an identification reader configured to acquire data representing an identifier from an identification tag; and
a controller coupled with the gas sensor and the identification reader, wherein, in response to the identification reader acquiring the data representing the identifier, the controller causes the gas sensor to measure a concentration of a gas in a test area.

2. The apparatus of claim 1, wherein the identification reader comprises an RFID antenna.

3. The apparatus of claim 1, wherein the identification reader comprises a bar-code scanner, a QR-code scanner, or a combination thereof.

4. The apparatus of claim 1, further comprising a shell body into which the identification reader and the controller are at least partially received, the shell body comprising a mounting device configured to be removably coupled with a mobile unit.

5. The apparatus of claim 4, wherein the shell body defines a recess therein, the apparatus further comprising one or more electrical contacts received into the recess and electrically coupled with the controller, the one or more electrical contacts being configured to electrically communicate with the mobile unit, when the mounting device is coupled therewith.

6. The apparatus of claim 5, wherein the recess extends at least partially around the mounting device.

7. The apparatus of claim 1, wherein the controller is configured to compare a gas concentration measurement taken by the gas sensor with a gas concentration threshold associated with the test area.

8. The apparatus of claim 7, wherein the controller is configured to select a type of gas to measure the gas concentration of, based on predetermined, location-specific information associated with the identifier.

9. A sensor system, comprising:
a mobile unit comprising a power source; and
a sensory head releasably coupled with the mobile unit, such that the sensory head is in electrical communication with the mobile unit so as to receive power from the power source and to provide a communication signal to the mobile unit, wherein the sensory head comprises:
a gas sensor;
an identification reader configured to acquire data representing an identifier from an identification tag; and
a controller coupled with the gas sensor and the identification reader, wherein, in response to the identification reader acquiring the data representing the identifier, the controller causes the gas sensor to measure a concentration of a gas in a test area.

10. The sensor system of claim 9, wherein the identification reader comprises a radio-frequency identification (RFID) tag reader, and the identification tag comprises an RFID tag.

11. The sensor system of claim 9, wherein the controller is configured to supply data representing the identifier to the mobile unit, and wherein the mobile unit is configured to determine location-specific information associated with the test area, based on the identifier.

12. The sensor system of claim 9, further comprising a remote device in communication with the mobile unit, wherein the controller is configured to supply data representing the identifier to the mobile unit, the mobile unit is configured to supply data representing the identifier to the remote device, and the remote device is configured to determine location-specific information related to the test area, based on the identifier.

13. The sensor system of claim 9, wherein the sensory head comprises a body shell in which the gas sensor, the identification reader, and the controller are at least partially received, wherein the body shell comprises a mounting device configured to releasably couple with the mobile unit.

14. The sensor system of claim 13, wherein the mounting device comprises a threaded shaft configured to be threaded into the mobile unit.

15. The sensor system of claim 14, wherein the body shell defines a recess extending at least partially around the mounting device, the sensory head further comprising one or more electrical contacts received into the recess and configured to electrically communicate with the mobile unit, when the sensory head is coupled with the mobile unit.

16. A method for measuring a gas concentration, comprising:
coupling a sensory head to a mobile unit such that the sensory head receives at least power from the mobile unit and supplies a communication signal to the mobile unit, wherein the sensory head includes a controller, an identification reader coupled with the controller, and a gas sensor coupled with the controller;
obtaining data representing an identifier from an identification tag positioned in, on, or proximal to a test area, using the identification reader; and
in response to obtaining the data representing the identifier, taking one or more gas concentration measurements using the gas sensor.

17. The method of claim 16, further comprising accessing location-specific information related to the test area based on the identifier.

18. The method of claim 17, further comprising determining a type of gas concentration to measure, based at least in part on the location-specific information.

19. The method of claim 17, further comprising:
determining a concentration threshold based at least in part on the location-specific information;
determining that a measured gas concentration exceeds the concentration threshold; and
indicating a hazard in response to determining that the measured gas concentration exceeds the concentration threshold.

20. The method of claim 17, further comprising detecting, using the controller, that the identifier has been obtained, wherein taking the one or more gas concentration measurements comprises sending a signal from the controller to the gas sensor when the controller detects the identifier has been obtained.
